(19) United States
(12) Patent Application Publication
(10) Pub. No.: US 2023/0090479 A1
(43) Pub. Date: Mar. 23, 2023

(54) COMPOSITION FOR FORMING ARTIFICIAL SKIN, AND USAGE METHOD THEREFOR

(71) Applicant: Shiseido Company, Ltd., Chuo-ku, Tokyo (JP)

(72) Inventors: Tomoko SATO, Tokyo (JP); Satoshi YAMAKI, Tokyo (JP); Hideo HATA, Tokyo (JP)

(73) Assignee: Shiseido Company, Ltd., Chuo-ku, Tokyo (JP)

(21) Appl. No.: 17/800,830

(22) PCT Filed: Mar. 10, 2021
(86) PCT No.: PCT/JP2021/009612

§ 371 Date: Aug. 18, 2022

Related U.S. Application Data
(60) Provisional application No. 62/990,541, filed on Mar. 17, 2020.

(51) Int. Cl. A61L 27/60; A61L 27/02; A61L 27/48

(52) U.S. Cl. CPC A61L 27/60; A61L 27/025; A61L 27/48

(57) ABSTRACT

There is provided a composition for forming an artificial skin that can shorten the cross-linking time while maintaining excellent application performance and coating performance.

A composition for forming an artificial skin of the present disclosure comprises a first agent and a second agent, wherein the first agent comprises: (a) a polymer A composed of one or more organopolysiloxanes containing at least two carbon-carbon double bonds or at least one carbon-carbon triple bond in the molecule; (b) a polymer B composed of one or more organopolysiloxanes containing at least two Si—H units in the molecule; and (c) a silicone having a viscosity of 1.5 mPa·s or less at 25° C., and wherein the second agent comprises a catalyst that promotes cross-linking of the polymer A and the polymer B.

### FIELD

The present disclosure relates to a composition for forming an artificial skin and a usage method therefor.

### BACKGROUND

In recent years, technologies related to artificial skin that can be used for damaged skin, or the like, have become known.

Patent Literature 1 discloses a method for producing a film containing fibers on the surface of a skin or a nail, comprising electrostatically spraying a composition A directly onto a human skin or nail, wherein the composition A contains (a) one or more types of volatile substances selected from alcohols and ketones, (b) water-insoluble polymer for fiber formation, and (c) from 0.2% by mass to 25% by mass of water, and wherein the mass ratio (b/c) of the ingredient (b) to the ingredient (c) is from 0.4 to 50 or less.

Patent Literature 2 discloses a body correcting formulation for application to a skin, comprising (a) a reactive reinforcing ingredient and (b) a cross-linking ingredient; wherein the cross-linking ingredient catalyzes cross-linking of the reactive reinforcing ingredient in situ, resulting in formation of a body correcting film on the skin, and resulting in a natural skin appearance when the film is applied to the body.

### CITATION LIST

### Patent Literature

[PTL 1] Japanese Unexamined Patent Publication No. 2018-177803
[PTL 2] Japanese Translation of PCT International Application Publication No. 2013-536861

### SUMMARY

### Technical Problem

A coating film as described in Patent Literature 1 is a special coating film containing fibers formed by electrostatic spray, and in order to form such a coating, it was necessary to use a dedicated and expensive electrostatic spray apparatus. A certain degree of skill was required to form a desired film at a desired location using electrostatic spray equipment.

On the other hand, a coating film as described in Patent Literature 2 does not require use of an electrostatic spray apparatus as described in Patent Literature 1, and can be applied and spread onto the skin of a body to form such a coating film. However, such a coating film requires a relatively long time for cross-linking, and therefore, it has been desired to shorten the cross-linking time.

Accordingly, a subject matter of the present disclosure is to provide a composition for forming an artificial skin that can shorten the cross-linking time while maintaining excellent application performance and coating performance.

### Solution to Problem

### <Aspect 1>

A composition for forming an artificial skin, comprising a first agent and a second agent, wherein

the first agent comprises: (a) a polymer A composed of one or more organopolysiloxanes containing at least two carbon-carbon double bonds or at least one carbon-carbon triple bond in the molecule; (b) a polymer B composed of one or more organopolysiloxanes containing at least two Si—H units in the molecule; and (c) a silicone having a viscosity of 1.5 mPa·s or less at 25° C., and wherein

the second agent comprises a catalyst that promotes cross-linking of the polymer A and the polymer B.

### <Aspect 2>

The composition according to Aspect 1, wherein the silicone is a polydimethylsiloxane having from 3 to 5 silicon atoms.

### <Aspect 3>

The composition according to Aspect 1 or 2, wherein the polymer A is at least one selected from the group consisting of vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinylmethylsiloxane homopolymer, vinyl T-structure polymer, monovinyl-terminated polydimethylsiloxane, vinylmethylsiloxane terpolymer, and vinylmethoxysilane homopolymer.

### <Aspect 4>

The composition according to any one of Aspects 1 to 3, wherein the polymer B is at least one selected from the group consisting of hydride-terminated polydimethylsiloxane, hydride-terminated polyphenyl-(dimethylhydrosiloxy)siloxane, hydride-terminated methylhydrosiloxane-phenylmethylsiloxane copolymer, trimethylsiloxy-terminated methylhydrosiloxane-dimethylsiloxane copolymer, polymethylhydrosiloxane, trimethylsiloxy-terminated polyethylhydrosiloxane, triethylsiloxane, methylhydrosiloxane-phenyloctylmethylsiloxane copolymer, and methylhydrosiloxane-phenyloctylmethylsiloxane terpolymer.

### <Aspect 5>

The composition according to any one of Aspects 1 to 4, wherein the viscosity of the first agent is 20,000 mPa·s or less.

### <Aspect 6>

The composition according to any one of Aspects 1 to 5, wherein the first agent comprises at least one selected from the group consisting of a fiber, a pigment, a dye, and a filler.

### <Aspect 7>

A composition according to any one of Aspects 1 to 6, wherein the second agent is free of a pigment, a dye and a filler.

### <Aspect 8>

A method for using the composition for forming an artificial skin according to any one of Aspects 1 to 7, wherein

after the first agent is applied to the surface of a body to form a first agent layer, a second agent is applied on the first agent layer and cross-linked to form an artificial skin, or

after the second agent is applied to the surface of the body to form a second agent layer, the first agent is applied on the second agent layer and cross-linked to form an artificial skin.

### <Aspect 9>

The method for using according to Aspect 8, wherein the cross-linking time is 30 seconds or less.

### Advantageous Effects of Invention

According to the present disclosure, a composition for forming an artificial skin that can shorten the cross-linking time while maintaining excellent application performance and coating performance can be provided.

### BRIEF DESCRIPTION OF DRAWINGS

FIG. 1 is a photograph of a composition for forming an artificial skin of one embodiment of the present disclosure containing fibers, applied to a skin.

FIG. 2 is a graph of the viscosity and the cross-linking time of silicone-based oils.

FIG. 3 is a graph of the weight loss rate, which is an indicator of the volatility of various oils.

### DESCRIPTION OF EMBODIMENTS

In the following, embodiments of the present disclosure will be described in detail. The present disclosure is not limited to the following embodiments, and can be implemented with a variety of variations within the scope of the main purpose of the invention.

The composition for forming an artificial skin of the present disclosure comprises a first agent and a second agent, the first agent comprises: (a) a polymer A composed of one or more organopolysiloxanes containing at least two carbon-carbon double bonds or at least one carbon-carbon triple bond in the molecule; (b) a polymer B composed of one or more organopolysiloxanes containing at least two Si—H units in the molecule; and (c) a silicone having a viscosity of 1.5 mPa·s or less at 25° C., and the second agent comprises a catalyst that promotes cross-linking of the polymer A and the polymer B.

The principle of action by which the composition for forming an artificial skin of the present disclosure can shorten the cross-linking time while maintaining excellent application performance and coating performance is believed to be, but not limited to, the following.

When a one-component composition without cross-linking reaction is used in forming an artificial skin, for example, blending a fast volatilizing agent as a diluent can shorten the drying time after skin application, thereby reducing the formation time of the artificial skin. However, the composition of the present disclosure requires application of a first agent containing the polymer A and the polymer B described above to a skin, followed by further application of a second agent containing a cross-linking accelerating catalyst thereto to cross-link the polymer A and the polymer B. The performance of such a two-component system involving drying and cross-linking reaction is considerably different from that of a simple one-component system without cross-linking reaction in terms of compatibility, cross-linking reactivity, or the like, and therefore a technique used in such a simple one-component system cannot be used as it is. For example, isododecane, a known volatile oil, could not contribute to shortening the cross-linking time of the composition of the present disclosure.

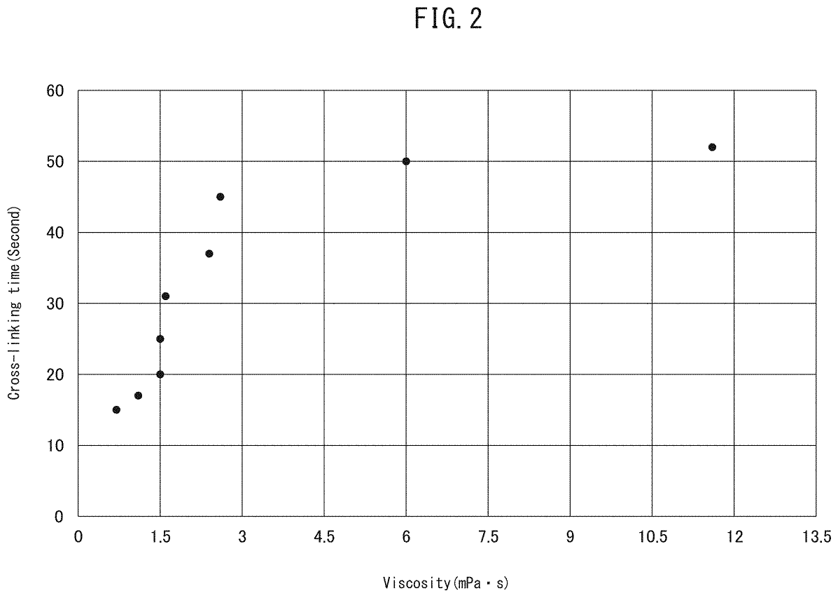

The present inventor has found that addition of a relatively low viscosity silicone oil to a first agent contributes considerably to shortening the cross-linking time of the composition of the present disclosure. FIG. 2 is a graph showing such a trend. In FIG. 2, for example, a silicone oil that shows a viscosity near 1.5 mPa·s and a cross-linking time near 30 seconds is a less volatile oil than the silicone oil to the right thereof, and such a silicone oil can shorten the cross-linking time of the composition of the present disclosure. In other words, the composition of the present disclosure is believed that the viscosity of an oil, especially silicone oil, rather than the volatility of the oil, contributes to shortening of cross-linking time.

Since silicones having such a specific viscosity have excellent compatibility with a polymer A and a polymer B in a first agent, and the polymer A and the polymer B can be mixed well in a balanced manner, it is also believed that a decrease in application performance due to an increase in viscosity and a decrease in coating performance due to non-uniform crosslinking formation can also be reduced or prevented.

The definitions of terms in the present disclosure are as follows.

In the present disclosure, “viscosity” refers to the measure of resistance of a fluid being deformed by either shear stress or tensile stress. The viscosities of a first agent and a second agent in a composition have an effect on the thickness, the spreadability, and the uniformity and/or the evenness of a layer formed on a substrate. A viscosity can be reported as either a dynamic viscosity (also known as absolute viscosity, typical units are Pas, Poise, P, cP.) or a kinematic viscosity (typical units are cm²/s, Stokes, St, cSt), and this kinematic viscosity is obtained by dividing the dynamic viscosity by the density of a fluid to be measured. The viscosity range of an ingredient disclosed herein is generally provided by the supplier of each ingredient as a unit of kinematic viscosity (for example, cSt), measured using a rheometer or a Cannon-Fenske tube viscometer, and the viscosity of a fluid can also be measured using, for example, a rheometer (for example, linear shear rheometer or dynamic shear rheometer) or a viscometer (also referred to as viscometer, for example, capillary viscometer or rotational viscometer).

The term “crosslinking” herein also encompasses the concept generally referred to as “curing”.

In the present disclosure, “body surface” means the skin surface of a body.

<<Composition for Forming Artificial Skin>>

The composition for forming an artificial skin (sometimes simply referred to as “composition”) of the present disclosure can reduce the cross-linking time while maintaining excellent application performance and coating performance.

In some embodiments, the application performance of a composition can be evaluated in terms of viscosity using a Type B viscometer (Vismetron, manufactured by Shibaura Systems Co., Ltd.). The viscosities of a first agent and a second agent in the composition of the present disclosure immediately after preparation, measured at 25° C., 60 rpm (rotor No. 3 or No. 4), can be, for example, 100 mPa·s or more, 500 mPa·s or more, 1,000 mPa·s or more, 2,000 mPa·s or more, 5,000 mPa·s or more, 7,500 mPa·s or more, 10,000 mPa·s or more, or 15,000 mPa·s or more, and can be 1,000,000 mPa·s or less, 750,000 mPa·s or less, 500,000 mPa·s or less, 250,000 mPa·s or less, 200,000 mPa·s or less, 175,000 mPa·s or less, 150,000 mPa·s or less, 125,000 mPa·s or less, 100,000 mPa·s or less, or 80,000 mPa·s or less. Among them, from the viewpoints of smooth application performance, prevention of dripping from a skin, and the like, a composition preferably has a viscosity of 20,000 mPa·s or less, 15,000 mPa·s or less, or 10,000 mPa·s or less and preferably has a viscosity of 3,000 mPa·s or more, 5,000 mPa·s or more, or 7,000 mPa·s or more.

In some embodiments, the viscosities of a first agent and a second agent in the composition of the present disclosure 2 weeks after preparation, measured at 25° C., 60 rpm (rotor No. 3), can be, from the viewpoints of smooth application performance, prevention of dripping from a skin, and the like, 50,000 mPa·s or less, 30,000 mPa·s or less, or 15,000 mPa·s or less, and preferably has a viscosity of 5,000 mPa·s or more, 7,000 mPa·s or more, or 10,000 mPa·s or more. Since silicone blended in the first agent has excellent compatibility with a polymer A and a polymer B, a considerable increase in viscosity can be reduced or prevented, even after 2 weeks.

In some embodiments, the coating performance of an artificial skin can be evaluated, for example, by the presence or absence of tearing of the artificial skin when pulled off from the skin. For example, when tearing of an applied artificial skin is 15% or less, 10% or less, or 5% or less of the total, it can be said that the coating performance is excellent. The lower limit of tearing is not particularly restricted, and can be defined, for example, as 0% or more, or more than 0%. In addition, the coating performance can be evaluated by tensile strength, elongation at break, or the like as described below.

The cross-linking time of the composition of the present disclosure can be evaluated using the following method.

After a first agent is applied on an aluminum substrate, a second agent is applied to the entire surface in such a manner that the second agent is stirred against the first agent, and a timer is activated. At some point in time, under an atmosphere of 30° C., a 1.5 cm×4 cm polypropylene sheet is placed on the surface to which the second agent is applied, then a 15 g weight is placed on the polypropylene sheet and allowed to stand for 2 seconds. The weight is then removed, followed by removal of the polypropylene sheet from the applied surface and observation of the presence of a composition adhering to the polypropylene sheet. The time at which the polypropylene sheet was applied when no composition was observed is defined as the “cross-linking time”.

In some embodiments, the composition of the present disclosure can achieve such a cross-linking time of 30 seconds or less, 25 seconds or less, or 20 seconds or less. The lower limit of the cross-linking time is not particularly limited, and can be, for example, 5 seconds or more, 8 seconds or more, or 10 seconds or more.

<First Agent>

A first agent constituting the composition of the present disclosure comprises: (a) a polymer A; (b) a polymer B; and (c) a silicone having a viscosity of 1.5 mPa·s or less at 25° C.

The first agent may be, for example, in anhydrous form, or in a form in which a polymer A and a polymer B are contained in a silicone, which is an oil, or in a form of an oil-in-water or water-in-oil emulsion, and from the viewpoint of drying and cross-linking after application of the first agent to a body surface, the first agent, it is advantageous for the first agent to be in anhydrous form.

When an agent is in an anhydrous form, a preservative against bacteria or mold is usually not needed, and the agent can be stored for a longer period of time than emulsions with similar ingredients. Herein, “anhydrous” in the present disclosure is intended to mean that a composition contains no water, as well as that the water content is low, namely 10% by mass or less, 5% by mass or less, 2% by mass or less, 1% by mass or less, or 0.1% by mass or less.

Since a first agent is applied to a body surface by application or the like, from the viewpoint of application performance, the first agent preferably has a glass transition temperature of body temperature or lower. For example, the glass transition temperature can be 37° C. or lower, 25° C. or lower, 10° C. or lower, or 0° C. or lower. The lower limit of the glass transition temperature is not particularly restricted, and can be, for example, −30° C. or higher, −20° C. or higher, or −10° C. or higher. Here, “glass transition temperature” refers to a temperature at which transition from a solid state to a liquid state occurs, and can be measured, for example, using a differential scanning calorimeter (DSC) in accordance with ASTM D3418-03.

((a) Polymer A)

A polymer A is composed of one or more organopolysiloxanes having at least two carbon-carbon double bonds or at least one carbon-carbon triple bond in the molecule, preferably one or more organopolysiloxanes having at least two alkenyl functional groups on average and a viscosity of from 10,000 to 2,000,000 cSt at 25° C. Herein, “carbon-carbon double bond” and “carbon-carbon triple bond” may be referred to simply as “double bond” and “triple bond”.

Such an organopolysiloxane may contain a double bond or a triple bond in a terminal unit of a polymer, in a non-terminal monomer unit of a polymer, or in a combination thereof, and particularly preferably in a non-terminal monomer unit of a polymer.

In one embodiment, double bond-containing monomer units in an organopolysiloxane may be, on average, 40 monomer units or more, 200 monomer units or more, 400 monomer units or more, 1,000 monomer units or more, or 2,000 monomer units or more apart.

In one embodiment, the amount of monomer units containing a double or a triple bond in an organopolysiloxane containing a double bond or a triple bond can be, for example, 0.01% by mass or more or 0.03% by mass or more, and can be 2% by mass or less or 0.6% by mass or less.

In one embodiment, the vinyl equivalent of organopolysiloxane containing a double bond or a triple bond can be, for example, 0.005 or more or 0.01 or more per kilogram, and can be 0.5 or less or 0.25 or less per kilogram. The approximate molar amount of double bonds or triple bonds in an organopolysiloxane can be calculated based on the average molecular weight of the organopolysiloxane. The average molecular weight or molecular mass of each ingredient disclosed herein is generally provided by the supplier of each ingredient and is expressed in daltons (Da) or in equivalent units of g/mol.

In one embodiment, a polymer A can have a viscosity of from 10,000 to 2,000,000 cSt at 25° C. The lower limit of the viscosity is preferably 20,000 cSt or more, 40,000 cSt or more, 60,000 cSt or more, 80,000 cSt or more, or 100,000 cSt or more, and more preferably 125,000 cSt or more or 150,000 cSt or more. The upper limit of the viscosity is preferably 1,000,000 cSt or less, 500,000 cSt or less, 450,000 cSt or less, 400,000 cSt or less, 350,000 cSt or less, 300,000 cSt or less, or 250,000 cSt or less, more preferably 200,000 cSt or less or 180,000 cSt or less, and further preferably 165,000 cSt or less.

In one embodiment, a polymer A can have an average molecular weight of from 60,000 Da to 500,000 Da. The lower limit of such an average molecular weight is preferably 72,000 Da or more, 84,000 Da or more, 96,000 Da or more, or 100,000 Da or more, and more preferably 140,000 Da or more or 150,000 Da or more. The upper limit of the average molecular weight is preferably 200,000 Da or less, 190,000 Da or less, 180,000 Da, or 170,000 Da or less, more preferably 160,000 Da or less, and further preferably 155,000 Da or less.

The polymer A can employ, for example, but not limited to, at least one selected from the group consisting of vinyl-terminated polydimethylsiloxane such as vinyl dimethicone, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinylmethylsiloxane homopolymer, vinyl T-structure polymer (branched vinyl polymer), monovinyl-terminated polydimethylsiloxane, vinylmethylsiloxane terpolymer, and vinylmethoxysilane homopolymer. Among them, vinyl-terminated polydimethylsiloxane is preferred, and vinyl dimethicone is more preferred.

The blending amount of a polymer A in a first agent may be adjusted according to the coating performance required, and there is no particular restriction. For example, the amount of a polymer A with respect to the total amount of the first agent can be 5% by mass or more, 10% by mass or more, 20% by mass or more, 30% by mass or more, 35% by mass or more, or 40% by mass or more, and can be 90% by mass or less, 80% by mass or less, 70% by mass or less, 60% by mass or less, 50% by mass or less, or 45% by mass or less.

((b) Polymer B)

A polymer B is composed of one or more organopolysiloxanes containing at least two Si—H units in the molecule, and preferably one or more organopolysiloxanes containing at least two Si—H units on average and having a viscosity of from 2 to 100,000 cSt at 25° C.

In one embodiment, an organopolysiloxane containing an Si—H unit may contain such an Si—H unit in a terminal unit of a polymer, in a non-terminal monomer unit of a polymer, or in a combination thereof, and particularly preferably in a non-terminal monomer unit of a polymer.

In one embodiment, Si—H unit-containing monomer units in an organopolysiloxane may be, on average, 1 monomer units or more, 2 monomer units or more, 5 monomer units or more, 10 monomer units or more, 20 monomer units or more, 40 monomer units or more, 200 monomer units or more, 400 monomer units or more, 1,000 monomer units or more, or 2,000 monomer units or more apart.

In one embodiment, the amount of monomer units containing an Si—H unit in an organopolysiloxane containing an Si—H unit can be 0.003% by mass or more or 0.01% by mass or more, and can be 50% by mass or less or 25% by mass or less.

In one embodiment, the Si—H content of organopolysiloxanes containing an Si—H unit can be 0.1 mmol/g or more, 0.5 mmol/g or more, or 1 mmol/g or more, and can be 20 mmol/g or less, 10 mmol/g or less, or 5 mmol/g or less. The approximate molar amount of Si—H units in an organopolysiloxane can be calculated based on the average molecular weight of the organopolysiloxane.

In one embodiment, a polymer B can have a viscosity of from 2 to 500,000 cSt at 25° C. The lower limit of such a viscosity is preferably 3 cSt or more, 4 cSt or more, or 12 cSt or more, and more preferably 40 cSt or more. The upper limit of the viscosity is preferably 200,000 cSt or less, 100,000 cSt or less, 50,000 cSt or less, 20,000 cSt or less, 10,000 cSt or less, 5,000 cSt or less, 2,000 cSt or less, or 1,000 cSt or less, and more preferably 500 cSt or less. The viscosity of a polymer B is particularly preferably in the range of from 45 to 100 cSt at 25° C.

In one embodiment, a polymer B can have an average molecular weight of from 400 to 500,000 Da. The lower limit of such an average molecular weight is preferably 500 Da or more, 800 Da or more, 1,200 Da or more, or 1,800 Da or more, and more preferably 2,000 Da or more. The upper limit of the average molecular weight is preferably 250,000 Da or less, 140,000 Da or less, 100,000 Da or less, 72,000 Da or less, 62,700 Da or less, 49,500 Da or less, 36,000 Da or less, or 28,000 Da or less, and more preferably 17,200 Da or less. The average molecular weight of a polymer B is particularly preferably in the range of from 2,200 Da to 6,000 Da.

The polymer B can employ, for example, but not limited to, at least one selected from the group consisting of hydride-terminated polydimethylsiloxane such as hydrogen dimethicone, hydride-terminated polyphenyl-(dimethylhydrosiloxy)siloxane, hydride-terminated methylhydrosiloxane-phenylmethylsiloxane copolymer, trimethylsiloxy-terminated methylhydrosiloxane-dimethylsiloxane copolymer, polymethylhydrosiloxane, trimethylsiloxy-terminated polyethylhydrosiloxane, triethylsiloxane, methylhydrosiloxane-phenyloctylmethylsiloxane copolymer, and methylhydrosiloxane-phenyloctylmethylsiloxane terpolymer. Among them, hydride-terminated polydimethylsiloxane is preferred, and hydrogen dimethicone is more preferred.

The blending amount of a polymer B in a first agent may be adjusted according to the coating performance required, and there is no particular restriction. For example, the amount of a polymer B with respect to the total amount of the first agent can be 1% by mass or more, 3% by mass or more, or 5% by mass or more, and can be 75% by mass or less, 60% by mass or less, 50% by mass or less, 40% by mass or less, 30% by mass or less, 20% by mass or less, or 10% by mass or less.

((c) Silicone Having Viscosity of 1.5 mPa·s or Less at 25° C.)

A silicone having a viscosity of 1.5 mPa·s or less at 25° C. (sometimes simply referred to as “silicone”) is included in a first agent as an oil. The viscosity of such a silicone is intended to be the viscosity measured by a falling-ball viscometer (manufactured by Anton Paar GmbH, AMVn automatic microviscometer) under 25° C. conditions. The viscosity of a silicone can be 1.5 mPa·s or less, 1.4 mPa·s or less, 1.3 mPa·s or less, or 1.2 mPa·s or less. The lower limit of viscosity of silicone is not particularly restricted, and can be, for example, 0.5 mPa·s or more, 0.7 mPa·s or more, or 1.0 mPa·s or more.

From the viewpoint of shortening drying time or the like, silicone is more preferably volatile. Such a volatility performance can be evaluated by the residual rate of silicone when 0.2 g of silicone is impregnated on a filter paper and left in a room temperature of 24° C. and humidity of 50% for 60 minutes. For example, the residual rate of silicone can be 20% or less, 15% or less, or 10% or less, and can be 0% or more or more than 0%.

The type of a silicone is not particularly restricted as long as the silicone has a viscosity as described above, and examples of such a silicone include polydimethylsiloxane having from 3 to 5 silicon atoms (sometimes referred to as “dimethyl silicone”). Such silicones can be used singly or in combination of two or more kinds.

The blending amount of silicone in a first agent may be adjusted according to the application performance, the cross-linking time, and the like, and is not particularly restricted. For example, the amount of silicone with respect to the total amount of the first agent can be 10% by mass or more, 15% by mass or more, 20% by mass or more, 25% by mass or more, 30% by mass or more, 35% by mass or more, or 40% by mass or more, and can be 70% by mass or less, 65% by mass or less, 60% by mass or less, 55% by mass or less, or 50% by mass or less.

<Second Agent>

A second agent constituting the composition of the present disclosure contains a catalyst that promotes cross-linking of the polymer A and polymer B described above.

A second agent may be, for example, in anhydrous form, or in a form containing a catalyst and an optional polymer C in oil, or in a form of an oil-in-water or water-in-oil emulsion, and from the viewpoint of applicability of a second agent to a surface to which a first agent is applied and cross-linking, and the like, it is advantageous for the second agent to be in a form of an oil-in-water or water-in-oil emulsion.

Since a second agent is applied by application or the like to a surface to which a first agent is applied, it is preferable for the second agent to have a glass transition temperature of body temperature or lower from the viewpoint of application performance. For example, the glass transition temperature can be 37° C. or lower, 25° C. or lower, 10° C. or lower, or 0° C. or lower. The lower limit of the glass transition temperature is not particularly restricted, and can be, for example, −30° C. or higher, −20° C. or higher, or −10° C. or higher.

(Catalyst)

Such a catalyst is not particularly restricted and can be, for example, any substance capable of causing, promoting, or initiating a physical and/or chemical cross-linking reaction. A catalyst optionally undergoes permanent physical and/or chemical changes during or at the end of a process.

The catalyst can include, but is not limited to, a metal catalyst capable of initiating and/or promoting cross-linking at or below body temperature, and examples thereof include a Group VIII metal catalyst such as a platinum catalyst, a rhodium catalyst, a palladium catalyst, a cobalt catalyst, a nickel catalyst, a ruthenium catalyst, an osmium catalyst, or an iridium catalyst and a Group IVA metal catalyst such as a germanium catalyst or a tin catalyst. Among them, a platinum catalyst, a rhodium catalyst, or a tin catalyst is preferred. These catalysts can be used singly or in combination of two or more kinds thereof.

Examples of a platinum catalyst include a platinum carbonyl cyclovinylmethylsiloxane complex, a platinum divinyltetramethyldisiloxane complex, a platinum cyclovinylmethylsiloxane complex, a platinum octanaldehyde/octanol complex, and another Pt(0) catalyst such as a Karstedt's catalyst, a platinum-alcohol complex, a platinum-alkoxide complex, a platinum-ether complex, a platinum-aldehyde complex, a platinum-ketone complex, a platinum-halogen complex, a platinum-sulfur complex, a platinum-nitrogen complex, a platinum-phosphorus complex, a platinum-carbon double-bond complex, a platinum-carbon triple-bond complex, a platinum-imide complex, a platinum-amide complex, a platinum-ester complex, a platinum-phosphate complex, a platinum-thiol ester complex, a platinum-isolated electron pair complex, a platinum-aromatic complex, or a platinum-t-electron complex, and a combination thereof.

Examples of a rhodium catalyst include tris(dibutyl sulfide)rhodium trichloride and rhodium trichloride hydrate.

Examples of a tin catalyst include tin (II) octanoate, tin (II) neodecanoate, dibutyltin diisooctylmaleate, di-n-butylbis(2,4-pentanedionate)tin, di-n-butylbutoxychlorotin, dibutyltin dilaurate, dimethyltin dineodecanoate, dimethylhydroxy(oleate)tin, and tin (II) oleate.

Among these catalysts, a platinum catalyst is more preferred, and a platinum divinyltetramethyldisiloxane complex is particularly preferred.

The blending amount of a catalyst in a second agent may be adjusted according to the coating performance or the like required, and is not particularly restricted. For example, the blending amount of a catalyst with respect to the total amount of the second agent can be 0.001% by mass or more, 0.005% by mass or more, or 0.01% by mass or more, and can be 1% by mass or less, 0.1% by mass or less, 0.05% by mass or less, or 0.03% by mass or less.

(Polymer C)

The second agent can optionally contain polymer C.

In one embodiment, a polymer C can have a viscosity of from 0.7 cSt to 10,000 cSt at 25° C. The lower limit of such a viscosity is preferably 1 cSt or higher, 6 cSt or higher, 10 cSt or higher, 20 cSt or higher, 50 cSt or higher, or 100 cSt or higher, and more preferably 200 cSt or higher. The upper limit of the viscosity is preferably 5,000 cSt or less, 4,000 cSt or less, 2,000 cSt or less, or 1,000 cSt or less, more preferably 500 cSt or less, and particularly preferably 250 cSt or less.

In one embodiment, a polymer C can have an average molecular weight of from 180 Da to 65,000 Da. The lower limit of such an average molecular weight is preferably 500 Da or more, 800 Da or more, 1,500 Da or more, 3,000 Da or more, or 6,000 Da or more, and more preferably 9,400 Da or more. The upper limit of the average molecular weight is preferably 50,000 Da or less, 45,000 Da or less, or 30,000 Da or less, more preferably 17,500 Da or less, and particularly preferably 10,000 Da or less.

Examples of the polymer C preferably include one or more organopolysiloxanes containing at least one alkenyl functional group on average and having a viscosity of from 0.7 to 10,000 cSt at 25° C.

Specifically, the polymer C can employ, for example, at least one selected from the group consisting of vinyl-terminated polydimethylsiloxane, vinyl-terminated diphenylsiloxane-dimethylsiloxane copolymer such as vinyl dimethicone, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethylsiloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinyl-terminated vinyl rubber, vinylmethylsiloxane homopolymer, vinyl T-structure polymer, vinyl Q-structured polymer, an unsaturated organic polymer (examples thereof include an unsaturated fatty alcohol, an unsaturated fatty acid, an unsaturated fatty ester, an unsaturated fatty amide, an unsaturated fatty urethane, an unsaturated fatty urea, ceramide, crocetin, lecithin, and sphingosine), monovinyl-terminated polydimethylsiloxane, vinylmethylsiloxane terpolymer, vinylmethoxysilane homopolymer, vinyl-terminated polyalkylsiloxane polymer, and vinyl-terminated polyalkoxysiloxane polymer. Among them, vinyl-terminated polydimethylsiloxane is preferred, and vinyl dimethicone is more preferred.

The blending amount of a polymer C in a second agent may be adjusted according to a required coating performance or the like, and is not particularly restricted. For example, the blending amount of a polymer C with respect to the total amount of a second agent can be 0.01% by mass or more, 0.1% by mass or more, 0.3% by mass or more, or 0.5% by mass or more, and can be 20% by mass or less, 15% by mass or less, or 10% by mass or less.

<Functional Group Ratios in Polymers A to C>

In one embodiment, the molar ratio of the Si—H functional group from a polymer B to the alkenyl functional group from a polymer A is preferably from 60:1 to 1:5, and more preferably from 45:1 to 15:1.

In one embodiment, the molar ratio of the Si—H functional group from a polymer B to the alkenyl functional group from a polymer C is preferably from 60:1 to 1:5, and more preferably from 45:1 to 15:1.

In one embodiment, the molar ratio of the alkenyl functional group from a polymer A to the alkenyl functional group from a polymer C is preferably from 100:1 to 1:100, and more preferably from 10:1 to 1:10.

<Optional Ingredient>

The composition of the present disclosure may contain one or more optional ingredients for a first agent and/or a second agent.

Examples of the optional ingredient include, but are not limited to, a feel modifier, a pressure sensitive adhesion modifier, a spreading promoter, a diluent, an adhesion modifier, an emulsifier, an emollient, a surfactant, a thickener, a solvent, a film-forming agent, a humectant, a preservative, a fiber, a pigment, a dye, a filler, a skin penetration enhancer, an optical modifier, a scattering agent, an adsorbent, a magnetic material, a gaseous transport modifier, a liquid transport modifier, a pH modifier, a sensitizing modifier, and an aesthetic modifier.

Examples of the emulsifier include alkoxydimethicone, alkyl dimethicone, amodimethicone, sulfodimethicone, phosphodimethicone, borodimethicone, halodimethicone, fluorodimethicone, chlorodimethicone, bromodimethicone, charged dimethicone, and a combination thereof.

Examples of the filler can include at least one selected from carbon, silver, mica, zinc sulfide, zinc oxide, titanium dioxide, aluminum oxide, clay, chalk, talc, calcite (such as CaCO₃), barium sulfate, zirconium dioxide, polymer beads, silica (such as fumed silica, silica acid, or anhydrous silica), silica aluminate, and calcium silicate, which may be surface-treated. Such a filler can improve the physical properties (such as strength) of a film (artificial skin), and can also serve as a viscosity modifier. Among the fillers, a surface-treated silica, such as a silica treated with a surface treatment agent such as hexamethyldisilazane, polydimethylsiloxane, hexadecylsilane, or methacrylsilane, is preferred. Fumed silica is also preferred, and for example, fumed silica surface treated with hexamethyldisilazane or the like can also suitably be used.

In one embodiment, a filler can have a specific surface area of from 50 to 500 m²/g. The specific surface area of a filler is preferably from 100 to 350 m²/g, and more preferably from 135 to 250 m²/g. The specific surface area of a filler can be calculated using the BET method.

In one embodiment, a filler can have an area circular equivalent particle diameter of from 1 nm to 20 μm. The area circular equivalent particle diameter of a filler is preferably from 2 nm to 1 μm, and more preferably from 5 nm to 50 nm. Here, the area circular equivalent particle diameter of a filler can be intended, for example, as the particle diameter when converted into a circular particle having the same area as the projected area of a filler particle observed with a transmission electron microscope. Such an area circular equivalent particle diameter can be defined as an average value of 10 or more particles.

When a filler is blended in a first agent, the blending amount of the filler with respect to the total amount of the first agent can be, for example, 1% by mass or more, 3% by mass or more, or 5% by mass or more, and can be 25% by mass or less, 15% by mass or less, or 10% by mass or less.

From the viewpoint of reinforcing properties of an artificial skin or the like, the mass ratio of the total amount of polymers A to C to a filler can be from 100:1 to 1:1, and preferably from 50:1 to 2:1, more preferably from 15:1 to 3:1, further preferably from 10:1 to 4:1, and particularly preferably from 5:1 to 9:1.

At least one selected from a pigment, a dye, and a filler among the optional ingredients is preferably blended in a first agent. In particular, in cases in which a pigment and a dye are blended into a second agent, when this second agent is applied to the surface to which a first agent is applied, the pigment or the dye may harden during the application, and the pigment or the dye may easily become localized, which may cause color unevenness. From the viewpoint of suppressing color unevenness, it is advantageous for a pigment and a dye to be blended in a first agent. A pigment, a dye, and a filler may be blended in a second agent to an extent that color unevenness does not occur, and it is advantageous for them not to be contained in the second agent.

In one embodiment, in the composition of the present disclosure, one or more agents can be further blended with respect to a first agent and/or a second agent. Examples of such an agent include a cosmetic agent, a therapeutic agent, a stimulant-response agent, and a drug delivery agent.

Examples of a suitable cosmetic agent include a moisturizing agent, a UV absorber, a skin protectant, a skin calming agent, a skin whitener, a skin brightener, a skin brightener, a skin softener, a skin smoothing agent, a skin bleaching agent, a skin exfoliator, a skin tightener, a beauty agent, a vitamin, an antioxidant, a cell signaling agent, a cell modulator, a cell interaction agent, a skin tanning agent, an anti-aging agent, an anti-wrinkle agent, a spot reducer, an α-hydroxy acid, a β-hydroxy acid, and a ceramide.

Examples of a suitable therapeutic agent include a pain reliever, an analgesic, an antipruritic agent, an antirrheumatic agent (such as beta-hydroxy acids, salicylic acid, benzoyl peroxide), an anti-inflammatory agent, an antihistamine, a corticosteroid, a NSAID (nonsteroidal anti-inflammatory drug), an antiseptic, an antibiotic, an antibacterial agent, an antifungal agent, an antiviral agent, an antiallergic agent, an anti-irritant agent, an insect repellent, a phototherapeutic agent, a blood coagulant, an anti-neoplastic agent, an immune system stimulant, an immune system suppressant, a coal tar, an anthralin, a fluocinonide, methotrexate, cyclosporine, pimecrolimus, tacrolimus, azathioprine, fluorouracil, a ceramide, a counterirritant, a skin-cooling compound.

Examples of a suitable agent include an antioxidant, a vitamin, a vitamin D₃ analog, a retinoid, a mineral, a mineral oil, Vaseline, a fatty acid, a plant extract, a polypeptide, an antibody, a protein, a sugar, a humectant, and an emollient.

<<Method for Using Composition for Forming Artificial Skin>>

The composition for forming an artificial skin of the present disclosure can be used, for example, for cosmetic or medical purposes. Herein, the method for using a composition for forming an artificial skin of the present disclosure does not encompass a method of operating, treating, or diagnosing a human being.

Specific examples of the method for using the composition for forming an artificial skin include: after the first agent is applied to the surface of a body to form a first agent layer, a second agent is applied on the first agent layer and cross-linked to form an artificial skin; and after the second agent is applied to the surface of the body to form a second agent layer, the first agent is applied on the second agent layer and cross-linked to form an artificial skin. From the viewpoint of obtaining a uniform artificial skin having little unevenness, as such a usage method, a method in which a first agent is applied to the body surface to form a first agent layer, and then a second agent is applied on this first agent layer and cross-linked to form an artificial skin is preferable. Here, with respect to the first agent and the second agent, the materials and the like described above can be used in the same manner.

This method may be performed once, or such a method may be performed on a formed artificial skin a plurality of times.

In some embodiments, methods for using a composition for forming an artificial skin of the present disclosure may also be utilized as a cosmetic method. The term “cosmetic method” means application of the composition for forming an artificial skin of the present disclosure to the body surface to form an artificial skin to beautify conditions of the body surface or a method thereof, which is different from a method of operating, treating, or diagnosing a human being.

The application method of a first agent or a second agent to a body surface or to a first agent layer or a second agent layer is not particularly restricted, and can employ, for example, means of spreading with a finger or the like, spray application, transfer, or the like.

In the method for using a composition of the present disclosure, since a silicone having a viscosity of 1.5 mPa·s or less at 25° C. is contained in a first agent, as described above, the cross-linking time can achieve 30 seconds or less, 25 seconds or less, or 20 seconds or less.

<Application Site>

The composition for forming an artificial skin of the present disclosure can be applied on the surface of a skin in any part of a body, namely, on any part of the body surface. For example, the composition can be applied to the skin surface of a face (a lip, an eye, a nose, a cheek, a forehead, or the like), a neck, an ear, a hand, an arm, a leg, a foot, a chest, a belly, a back, or the like, as appropriate. Here, the skin also encompasses a nail or the like, which is formed as a result of a change of keratin of the epidermis of a skin and hardened.

<< Artificial Skin>>

<Thickness>

The thickness of an artificial skin prepared using the composition for forming an artificial skin of the present disclosure described above is not particularly limited, and can be adjusted as appropriate, taking into consideration, for example, breathability, invisibility, compressibility, and closure to a skin. The thickness of an artificial skin can be, for example, 0.5 μm or more, 1 μm or more, 10 μm or more, 30 μm or more, or 40 μm or more. The upper limit of the thickness is not particularly limited, and can be, for example, 150 μm or less, 100 μm or less, 90 μm or less, 80 μm or less, 70 μm or less, 60 μm or less, or 50 μm or less. Here, the thickness can be defined as an average value calculated by measuring the thickness of any portion of an artificial skin five times using a High-Accuracy Digimatic Micrometer (MDH-25 MB, manufactured by Mitutoyo Corporation).

<Performance>

An artificial skin prepared from the composition for forming an artificial skin of the present disclosure can provide excellent results for a variety of performances such as those shown below.

(Adhesive Strength)

In some embodiments, an obtained artificial skin can exhibit favorable adhesive strength to a body surface. Such adhesive strength can be evaluated alternatively by the adhesive strength of an artificial skin applied on a polypropylene substrate. The adhesive strength of an artificial skin on a polypropylene substrate can achieve 2 N/m or more, 5 N/m or more, 8 N/m or more, 10 N/m or more, or 15 N/m or more. The upper limit of such an adhesive strength is not particularly limited, and, for example, the upper limit can be, from the viewpoint of peelability from a skin or the like, 200 N/m or less, 100 N/m or less, 80 N/m or less, 50 N/m or less, or 30 N/m or less. Here, the adhesive strength can be measured using an Instron device in accordance with the ASTM C794 peel-off adhesion test.

(Tensile Strength)

In some embodiments, an obtained artificial skin can exhibit favorable tensile strength. The tensile strength of an artificial skin can achieve 0.05 MPa or more, 0.10 MPa or more, 0.20 MPa or more, or 0.50 MPa or more. The upper limit of the tensile strength is not particularly restricted, and can be, for example, 5.0 MPa or less, 3.0 MPa or less, 2.0 MPa or less, or 1.0 MPa or less. Here, the tensile strength can be measured using an Instron device in accordance with ASTM D5083 elongation tensile test.

(Elongation at Break)

In some embodiments, an obtained artificial skin can exhibit favorable elongation at break. The elongation at break of an artificial skin can achieve 25% or more, 50% or more, 100% or more, 200% or more, or 400% or more. The upper limit of the elongation at break is not particularly restricted, and can be, for example, 1,500% or less, 1,200% or less, 1,000% or less, 800% or less, or 600% or less. Here, the elongation at break can be measured using an Instron device in accordance with ASTM D5083 elongation tensile test.

(Oxygen Permeability Coefficient)

In some embodiments, an obtained artificial skin can exhibit a favorable oxygen permeability coefficient. In an artificial skin having a thickness of 300 μm, the oxygen permeability coefficient of the artificial skin can achieve 5×10⁻⁹ cm³/(cm²·s) or more, 5×10⁻⁷ cm³/(cm²·s) or more, or 5×10⁻⁵ cm³/(cm²·s) or more. The upper limit of the oxygen permeability coefficient is not particularly restricted, and can be, for example, 5 cm³/(cm²·s) or less, 0.5 cm³/(cm²·s) or less, 5×10⁻² cm³/(cm²·s) or less, 5×10⁻³ cm³/(cm²·s) or less, or 5×10⁻⁴ cm³/(cm²·s) or less. Here, the oxygen permeability coefficient can be measured using a Mocon device in accordance with the ASTM F2622 oxygen gas permeability coefficient test for plastic films and sheets.

(Water Vapor Permeability Coefficient)

In some embodiments, an obtained artificial skin can exhibit a favorable water vapor permeability coefficient. In an artificial skin having a thickness of 300 μm, the water vapor permeability coefficient of the artificial skin can achieve 1×10⁻⁹ cm³/(cm²·s) or more, 1×10⁻⁸ cm³/(cm²·s) or more, or 1×10⁻⁷ cm³/(cm²·s) or more. The upper limit of the water vapor permeability coefficient is not particularly restricted, and can be, for example, 1.5×10⁻¹ cm³/(cm²·s) or less, 1.5×10⁻² cm³/(cm²·s) or less, 1×10⁴ cm³/(cm²·s) or less, 1×10⁻⁵ cm³/(cm²·s) or less, or 1×10⁻⁶ cm³/(cm²·s) or less. Here, the water vapor permeability coefficient can be measured using a Mocon device in accordance with the ASTM F1249 water vapor permeability coefficient test for plastic films and sheets.

<<Kit Containing Composition for Forming Artificial Skin>>

The composition for forming an artificial skin of the present disclosure may be provided as a kit containing a first agent and a second agent constituting such a composition. In addition to the first agent and the second agent, the kit may also contain an optional member, for example, to facilitate application of the first agent or the like to a body surface or to further apply cosmetics.

Examples of such an optional member include a usage instruction, a brush, a cotton swab, a cutter, scissors, a gloss, a lipstick, a nail polish, a foundation, a cleanser for removing an artificial skin from a body surface, and a mirror. Here, “usage instruction” can encompass, in addition to a general usage instruction attached to a kit in the form of a document, for example, an instruction printed on a packaging container that contains the kit or on a packaging container such as a tube or the like that injects the first agent or the like.

In one embodiment, in order to prevent contact between the first agent and the second agent, for example, the kit may be configured in such a manner that these agents are packaged in separate containers or compartments and are applied one at a time, or mixed together before or at the time of use.

### EXAMPLES

The present invention will be described in more detail by way of Examples below, but is not limited thereto. Unless otherwise noted, the blending amounts are shown below in % by mass.

### Examples 1 to 4 and Comparative Examples 1 to 7

A first agent was prepared by uniformly mixing a polymer A, a polymer B, an oil, and a filler of each of the formulations shown in Table 1. The viscosity of the first agent after two weeks and the viscosity of each oil used in the first agent were evaluated by the method described below, and the results are shown in Table 1. The residual rate, which is an indicator of the volatility of each oil used in the first agent, was evaluated by the method described below, and the results are also shown in Table 1.

According to the composition in Table 2, an aqueous phase was prepared by uniformly mixing ingredients other than platinum catalyst dispersed in silicone oil and potassium hydroxide dissolved in some water, and then a second agent was prepared by mixing the platinum catalyst dispersed in silicone oil into the aqueous phase and further mixing a potassium hydroxide solution for neutralization. The prepared first agent and second agent were used to evaluate the cross-linking time of the compositions by the method described below. The results are shown in Table 1.

A graph of the relationship between the oil viscosity of silicone oils, or a mixture of dimethyl silicone (dimethicone) and trisiloxane, diphenylsiloxyphenyl trimethicone, five dimethicones of different viscosities, and caprylyl methicone and the cross-linking time of the compositions in Examples and each of Comparative Examples using these oils in Table 1 is shown in FIG. 2.

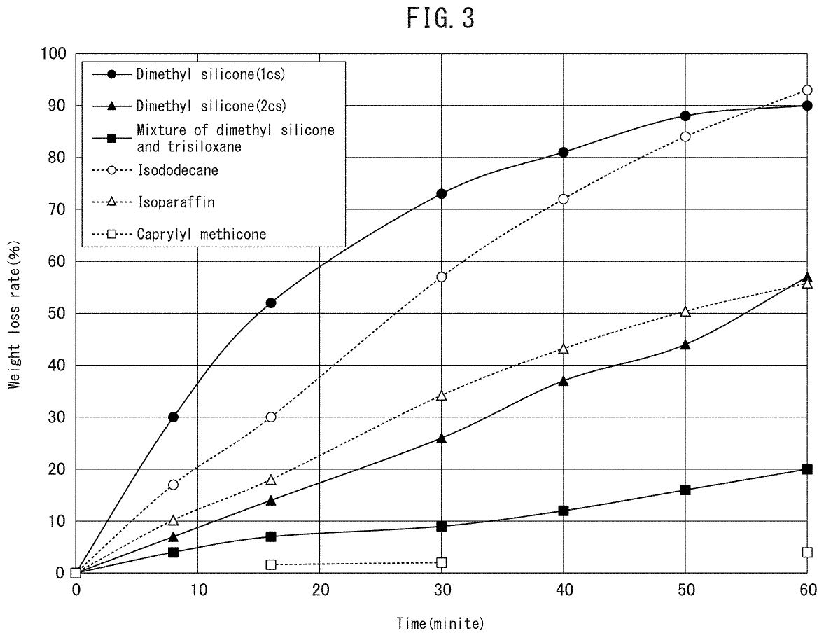

For reference, FIG. 3 shows graphs on the weight loss rate, which serves as an indicator of the volatility of two dimethicone products of different viscosities, a mixture of dimethyl silicone (dimethicone) and trisiloxane, isododecane, isoparaffin, and caprylyl methicone. The weight loss rate was determined in the same manner as the evaluation method for the residual rate of oil, described below.

<Evaluation Method>

(Evaluation of Viscosity)

The viscosity of the first agent was evaluated at 25° C. using a Type B viscometer (Vismetron, manufactured by Shibaura Systems Co., Ltd.) under the conditions of rotor number 3 or 4 and 60 rpm, and the viscosity of oil was evaluated at 25° C. using a falling-ball viscometer (manufactured by Anton Paar, AMVn automatic micro viscometer).

(Evaluation of Cross-Linking Time)

After 71 μl of a first agent was applied on an aluminum substrate, 100 μl of a second agent was applied to the entire surface in such a manner that the second agent was stirred against the first agent, and a timer was activated. At some point in time, under an atmosphere of 30° C., a 1.5 cm×4 cm polypropylene sheet was placed on the surface to which the second agent was applied, then a 15 g weight was placed on the polypropylene sheet and allowed to stand for 2 seconds. The weight was then removed, followed by removal of the polypropylene sheet from the applied surface and observation of the presence of a composition adhering to the polypropylene sheet. The time at which the polypropylene sheet was applied when no composition was observed is defined as the “cross-linking time”.

(Evaluation of Oil Residual Rate (Volatility))

On a filter paper placed on an electronic balance, 0.2 g of each oil listed in Table 1 was dropped onto the filter paper and left in an environment of 24° C. at room temperature and 50% humidity for 60 minutes, after which the weight of the oil was measured and the weight loss rate (%) of the oil was obtained from Formula 1 below. This was introduced into Formula 2 to calculate the oil residual rate (%), which serves as an indicator of volatility:

Weight loss rate of oil (%)=(0.2 (g)−oil content after 60 minutes (g))×100/0.2 (g) Formula 1

Residual rate of oil (%)=100−Weight loss rate of oil Formula 2

TABLE 1
Formulation of first agent, and evaluation result*

| | | Compar- | Compar- | Compar- | Compar- | Compar- | Compar- |
|---|---|---|---|---|---|---|---|
| | | ative | ative | ative | ative | ative | ative |
| Ingredient | Ingredient name | Example 1 | Example 2 | Example 3 | Example 4 | Example 5 | Example 6 |
| Polymer A | Vinyl dimethicone ¹⁾ | 53.0 | 53.0 | 53.0 | 53.0 | 53.0 | 53.0 |
| Polymer B | Hydrogen dimethicone ²⁾ | 13.0 | 13.0 | 13.0 | 13.0 | 13.0 | 13.0 |
| Filler | Silylated silica ³⁾ | 9.0 | 9.0 | 9.0 | 9.0 | 9.0 | 9.0 |
| | Graphene | — | — | — | — | — | — |
| Oil | Mixture of dimethicone | 25.0 | — | — | — | — | — |
| | and trisiloxane ⁴⁾ | | | | | | |
| | Glyceryl | — | 25.0 | — | — | — | — |
| | tri-2-ethylhexanoate | | | | | | |
| | Diphenylsiloxyphenyl | — | — | 25.0 | — | — | — |
| | trimethicone | | | | | | |
| | Dimethicone (6 cs) | — | — | — | 25.0 | — | — |
| | Caprylyl methicone | — | — | — | — | 25.0 | — |
| | Dimethicone (2 cs) | — | — | — | — | — | 25.0 |
| | Dimethicone (1.5 cs) | — | — | — | — | — | — |
| | Dimethicone (1 cs) | — | — | — | — | — | — |
| | Dimethicone (0.65 cs) | — | — | — | — | — | — |
| | Isododecane | — | — | — | — | — | — |
| Total | | 100 | 100 | 100 | 100 | 100 | 100 |
| Evaluation | Viscosity (mPa · s) of | 17,200 | 68,100 | 42,800 | 17,200 | 16,500 | 16,500 |
| | first agent after 2 weeks | | | | | | |
| | Viscosity (mPa · s) of oil | 1.6 | 25.5 | 11.6 | 6.0 | 2.6 | 2.4 |
| | Cross-linking time (secs.) | 31 | 65 | 52 | 50 | 45 | 37 |
| | Residual rate (%) of oil ** | 80 | 100 | 100 | 100 | 96 | 43 |

| | | | | | Compar- | |
|---|---|---|---|---|---|---|
| | | Exam- | Exam- | Exam- | ative | Exam- |
| Ingredient | Ingredient name | ple 1 | ple 2 | ple 3 | Example 7 | ple 4 |
| Polymer A | Vinyl dimethicone ¹⁾ | 53.0 | 53.0 | 53.0 | 53.0 | 40.0 |
| Polymer B | Hydrogen dimethicone ²⁾ | 13.0 | 13.0 | 13.0 | 13.0 | 10.0 |
| Filler | Silylated silica ³⁾ | 9.0 | 9.0 | 9.0 | 9.0 | 5.0 |
| | Graphene | — | — | — | — | — |
| Oil | Mixture of dimethicone | — | — | — | — | — |
| | and trisiloxane ⁴⁾ | | | | | |
| | Glyceryl | — | — | — | — | — |
| | tri-2-ethylhexanoate | | | | | |
| | Diphenylsiloxyphenyl | — | — | — | — | — |
| | trimethicone | | | | | |
| | Dimethicone (6 cs) | — | — | — | — | — |
| | Caprylyl methicone | — | — | — | — | — |
| | Dimethicone (2 cs) | — | — | — | — | 25.0 |
| | Dimethicone (1.5 cs) | 25.0 | — | — | — | — |
| | Dimethicone (1 cs) | — | 25.0 | — | — | — |
| | Dimethicone (0.65 cs) | — | — | 25.0 | — | 20.0 |
| | Isododecane | — | — | — | 25.0 | — |
| Total | | 100 | 100 | 100 | 100 | 100 |
| Evaluation | Viscosity (mPa · s) of | 14,500 | 13,000 | 10,000 | 12,000 | 14,000 |
| | first agent after 2 weeks | | | | | |
| | Viscosity (mPa · s) of oil | 1.5 | 1.1 | 0.7 | 1.7 | 1.5 |
| | Cross-linking time (secs.) | 25 | 17 | 15 | 57 | 20 |
| | Residual rate (%) of oil ** | 28 | 10 | 10 | 7 | 14 |

*A second agent of the formula shown in Table 2 was used as the second agent.
** Indicates the residual rate for the oil content of the formulation.
¹⁾ Andisil (trademark) VS 165,000 (manufactured by AB Specialty Silicones Co., Ltd.)
²⁾ Andisil (trademark) XL-11 (manufactured by AB Specialty Silicones Co., Ltd.)
³⁾ Aerosil (trademark) r812s (manufactured by EVONIC Corporation)
⁴⁾ XIAMETER (trademark) PMX-1184 Silicone Fluid (Dow Chemical Company)

TABLE 2
Formulation of second agent

| | | Blending |
|---|---|---|
| Ingredient | Ingredient name | amount |
| Water | Purified water | Remainder |
| Moisturizing | 1,3-Butylene glycol | 10.0 |
| agent | Glycerol | 4.0 |
| Surfactant | Carboxy vinyl polymer ¹⁾ | 0.2 |
| Catalyst | Platinum catalyst | Appropriate |
| | dispersed in silicone oil ²⁾ | amount |
| Neutralizer | Potassium hydroxide | 0.075 |
| Filler | Nylon particles | 7.0 |
| Organic solvent | Phenoxyethanol | 0.5 |
| | Ethanol | 10.0 |

| | |
|---|---|
| Total | 100 |

¹⁾ Carbopol (trademark) 980 (manufactured by AB Specialty Silicones Co., Ltd.)
²⁾ C1142AF

<Result>

As can be seen from the results in FIG. 2, it was confirmed that when the oil blended in the first agent is a silicone-based oil, the cross-linking time is reduced as the viscosity of the oil decreases.

For example, comparing Example 2 and Comparative Example 7 in Table 1, isododecane in Comparative Example 7 had a cross-linking time more than three times longer than dimethicone in Example 2, even though they had similar viscosity and volatility performance (residual rate). As can be seen from these results, it was confirmed that in order to shorten the cross-linking time in compositions containing a first agent containing specific polymers A and B and a second agent containing a catalyst that promotes cross-linking, it is important to employ silicones with a specific viscosity as the oil content in the first agent, rather than just using an oil having a fast volatilization rate.

### Examples 5 to 8

The dispersion of fibers in a system in which fibers were blended and the formability of an artificial skin were evaluated.

TABLE 3
Formulation of first agent containing fibers

| Ingre- | Ingredient | Exam- | Exam- | Exam- | Exam- |
|---|---|---|---|---|---|
| dient | name | ple 5 | ple 6 | ple 7 | ple 8 |
| Poly- | Vinyl | 35.0 | 35.0 | 40.0 | 40.0 |
| mer A | dimethicone ¹⁾ | | | | |
| Poly- | Hydrogen | 10.0 | 10.0 | 10.0 | 10.0 |
| mer B | dimethicone ²⁾ | | | | |
| Filler | Silylated | 5.0 | 5.0 | 5.0 | 5.0 |
| | silica ³⁾ | | | | |
| Oil | Dimethicone | 44.0 | 44.0 | 44.0 | 44.0 |
| | (1cs)⁴⁾ | | | | |
| Fiber | Cellulose fiber | 1.0 | — | — | — |
| | Polyester fiber | — | 1.0 | — | — |
| | Nylon 6 | — | — | 1.0 | — |
| | (length; 4 mm) | | | | |
| | Nylon 6 | — | — | — | 1.0 |
| | (length; 1 mm) | | | | |

| | | | | |
|---|---|---|---|---|
| Total | 100 | 100 | 100 | 100 |

¹⁾ Andisil (trademark) VS 165,000 (manufactured by AB Specialty Silicones Co., Ltd.)
²⁾ Andisil (trademark) XL-11 (AB Specialty Silicones Co., Ltd.)
³⁾ Aerosil (trademark) r812s (manufactured by EVONIC Corporation)
⁴⁾Wacker (trademark) Silicone 1 plus (manufactured by wacker Corporation)

<Results>

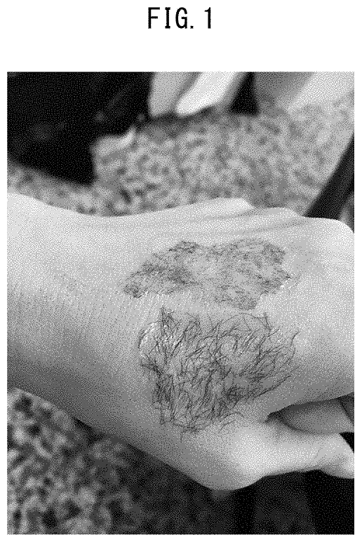

After preparing the first agent containing fibers according to the composition in Table 3, this first agent was applied to a skin, and the second agent described above was applied on top thereof in such a manner that the second agent was stirred against the first agent, and it was confirmed that favorable artificial skin was formed in all cases of Examples 5 to 8. Here, the lower portion on the back of the hand in FIG. 1 is the artificial skin prepared using the first agent in Example 7, and the upper portion on the back of the hand in FIG. 1 is the artificial skin prepared using the first agent in Example 8. In both cases, as shown in FIG. 1, the blended fibers showed favorable dispersion in the artificial skin.

### Formulation Example of First Agent and Second Agent

The following are formulation examples of the first agent and the second agent of the composition for forming an artificial skin of the present disclosure in Tables 4-8, but the first agent and the second agent of the composition for forming an artificial skin of the present disclosure are not limited to these examples.

TABLE 4
Formulation Example of First Agent

| | | Formu- | Formu- | Formu- |
|---|---|---|---|---|
| | | lation | lation | lation |
| Ingre- | Ingredient | exam- | exam- | exam- |
| dient | name | ple 1 | ple 2 | ple 3 |
| Poly- | Vinyl | 42.0 | 45.0 | 40.0 |
| mer A | dimethicone ¹⁾ | | | |
| Poly- | Hydrogen | 8.0 | 10.0 | 10.0 |
| mer B | dimethicone ²⁾ | | | |
| Filler | Silylated | — | — | 5.0 |
| | silica ³⁾ | | | |
| | Graphene | 7.0 | 7.0 | — |
| Oil | Mixture of | 20.0 | — | — |
| | dimethicone and | | | |
| | trisiloxane ⁴⁾ | | | |
| | Dimethicone | — | 18.0 | — |
| | (2cs) | | | |
| | Dimethicone | — | 20.0 | — |
| | (1.5cs) | | | |
| | Dimethicone | 23.0 | — | 20.0 |
| | (1cs) | | | |
| | Isododecane | — | — | 5.0 |

| | | | |
|---|---|---|---|
| Total | 100 | 100 | 100 |

¹⁾ Andisil (trademark) VS 165,000 (manufactured by AB Specialty Silicones Co., Ltd.)
²⁾ Andisil (trademark) XL-11 (manufactured by AB Specialty Silicones Co., Ltd.)
³⁾ Aerosil (trademark) r812s (manufactured by EVONIC Corporation)
⁴⁾ XIAMETER (trademark) PMX-1184 Silicone Fluid (Dow Chemical Company)

TABLE 5
Formulation example of first agent

| | | Formulation | Formulation | Formulation | Formulation | Formulation |
|---|---|---|---|---|---|---|
| Ingredient | Ingredient name | example 4 | example 5 | example 6 | example 7 | example 8 |
| Polymer A | Vinyl dimethicone ¹⁾ | 25.0 | 20.0 | 20.0 | 20.0 | 20.0 |
| Polymer B | Hydrogen dimethicone ²⁾ | 10.0 | 5.0 | 10.0 | 10.0 | 10.0 |
| Surfactant | PEG-10 dimethicone and | 1.0 | 1.0 | 1.0 | 1.0 | 1.0 |
| | tocopherol ³⁾ | | | | | |
| Water | Purified water | 40.0 | 40.0 | 40.0 | 35.0 | 30.0 |
| Salt | Sodium chloride | 1.0 | 1.0 | 1.0 | 5.0 | 10.0 |
| Moisturizing | Butylene glycol | 1.0 | 1.0 | 1.0 | 1.0 | 1.0 |
| agent | | | | | | |
| Organic | Phenoxyethanol | 0.5 | 0.5 | 0.5 | 0.5 | 0.5 |
| solvent | | | | | | |
| Oil | Dimethicone (6 cs) | 4.0 | — | — | 2.0 | — |
| | Isododecane | — | 4.0 | — | — | 5.0 |
| | Dimethicone (2 cs) | — | — | 4.0 | 5.0 | — |
| | Dimethicone (1.5 cs) | Remainder | Remainder | Remainder | Remainder | Remainder |

| | | | | | |
|---|---|---|---|---|---|
| Total | 100 | 100 | 100 | 100 | 100 |

¹⁾ Andisil (trademark) VS 165,000 (manufactured by AB Specialty Silicones Co., Ltd.)
²⁾ Andisil (trademark) XL-11 (manufactured by AB Specialty Silicones Co., Ltd.)
³⁾ KF-6017P (manufactured by Shin-Etsu Chemical Co., Ltd.)

TABLE 6
Formulation example of second agent

| | | Formulation | Formulation |
|---|---|---|---|
| Ingredient | Ingredient name | example 9 | example 10 |
| Water | Purified water | Remainder | Remainder |
| Moisturizing | Trehalose | 3.0 | — |
| agent | PEG-1500 | — | 5.0 |
| Surfactant | Polyether | — | 0.4 |
| | modified | | |
| | silicone ¹⁾ | | |
| | Carboxy vinyl | 0.2 | 0.2 |
| | polymer ²⁾ | | |
| Catalyst | Platinum catalyst | 1.3 | 1.3 |
| | dispersed in | | |
| | silicone oil ³⁾ | | |
| Neutralizer | Potassium | Appropriate | Appropriate |
| | hydroxide | amount | amount |
| Organic | Ethanol | 10.0 | 10.0 |
| solvent | Phenoxyethanol | 0.5 | 0.5 |
| Filler | Nylon-12 ⁴⁾ | 8.0 | 8.0 |

| | | |
|---|---|---|
| Total | 100 | 100 |

¹⁾ KF-6011P (manufactured by Shin-Etsu Chemical Co., Ltd.)
²⁾ Carbopol (trademark) 980 (manufactured by AB Specialty Silicones Co., Ltd.)
³⁾ C1142AF
⁴⁾ SP-500 (manufactured by Toray Industries, Inc.)

TABLE 7
Formulation example of second agent

| | | Formulation | Formulation | Formulation |
|---|---|---|---|---|
| Ingredient | Ingredient name | example 11 | example 12 | example 13 |
| Water | Purified water | Remainder | Remainder | Remainder |
| Moisturizing | Glycerol | 3.0 | — | — |
| agent | | | | |
| Surfactant | Polyether modified silicone ¹⁾ | 0.5 | — | — |
| | Carboxy vinyl polymer ²⁾ | 0.2 | 0.2 | 0.2 |
| Catalyst | Platinum catalyst dispersed in silicone oil ³⁾ | 1.3 | 1.3 | 1.3 |
| Neutralizer | Potassium hydroxide | Appropriate | Appropriate | Appropriate |
| | | amount | amount | amount |
| Organic | Ethanol | 10.0 | 10.0 | 10.0 |
| solvent | Phenoxyethanol | 0.5 | 0.5 | 0.5 |
| Coating film | Alkyl acrylate/vinyl acetate copolymer | 10.0 | — | — |
| agent | emulsion ⁴⁾ | | | |
| | Silicone/polyether polyurethane resin water | — | 10.0 | — |
| | dispersion ⁵⁾ | | | |
| | Alkyl acrylate ester copolymer aqueous emulsion | — | — | 10.0 |
| | polyurethane resin water dispersion ⁶⁾ | | | |

| | | | |
|---|---|---|---|
| Total | 100 | 100 | 100 |

¹⁾ KF-6012 (manufactured by Shin-Etsu Chemical Co., Ltd.)
²⁾ Carbopol (trademark) 980 (manufactured by AB Specialty Silicones Co., Ltd.)
³⁾ C1142AF
⁴⁾ VINYSOL (trademark) 2140L (manufactured by DAIDO CHEMICAL Co., Ltd.)
⁵⁾ YODOSOL (trademark) PUD (manufactured by Akzo Nobel)
⁶⁾ YODOSOL (trademark) GH34F (manufactured by Akzo Nobel)

TABLE 8
Formulation example of second agent

| | | Formulation | Formulation | Formulation |
|---|---|---|---|---|
| Ingredient | Ingredient name | example 14 | example 15 | example 16 |
| Water | Purified water | Remainder | Remainder | Remainder |
| Moisturizing | Glycerol | 3.0 | — | 2.0 |
| agent | 1,3-butylene glycol | — | 5.0 | 3.0 |
| Oil | Dimethicone (2cs) | 20.0 | 20.0 | 30.0 |
| Surfactant | Cetyl-PEG/PPG-10/1 dimethicone ¹⁾ | 1.0 | 1.0 | 1.0 |
| | (Dimethicone/(PEG-10/15)) | 10.0 | 10.0 | 10.0 |
| | crosspolymer ²⁾ | | | |
| Catalyst | Platinum catalyst dispersed in silicone | 2.0 | 2.0 | 2.0 |
| | oil ³⁾ | | | |
| Organic solvent | Ethanol | 10.0 | 10.0 | 5.0 |
| | Phenoxyethanol | 1.0 | 1.0 | 1.0 |
| Powder | (Vinyl dimethicone/methicone | 3.0 | 5.0 | 3.0 |
| | silsesquioxane) crosspolymer ⁴⁾ | | | |

| | | | |
|---|---|---|---|
| Total | 100 | 100 | 100 |

¹⁾ KF-6048 (manufactured by Shin-Etsu Chemical Co., Ltd.)
²⁾ KSG-240 (manufactured by Shin-Etsu Chemical Co., Ltd.)
³⁾ C1142AF
⁴⁾ KSP-100 (manufactured by Shin-Etsu Chemical Co., Ltd.)

1. A composition for forming an artificial skin, comprising a first agent and a second agent, wherein
the first agent comprises: (a) a polymer A composed of one or more organopolysiloxanes containing at least two carbon-carbon double bonds or at least one carbon-carbon triple bond in the molecule; (b) a polymer B composed of one or more organopolysiloxanes containing at least two Si—H units in the molecule; and (c) a silicone having a viscosity of 1.5 mPa·s or less at 25° C., and wherein the second agent comprises a catalyst that promotes cross-linking of the polymer A and the polymer B.

2. The composition according to claim 1, wherein the silicone is a polydimethylsiloxane having from 3 to 5 silicon atoms.

3. The composition according to claim 1, wherein the polymer A is at least one selected from the group consisting of vinyl-terminated polydimethylsiloxane, vinyl-terminated diphenylsiloxane-dimethylsiloxane copolymer, vinyl-terminated polyphenylmethylsiloxane, vinylphenylmethyl-terminated vinylphenylsiloxane-phenylmethylsiloxane copolymer, vinyl-terminated trifluoropropylmethylsiloxane-dimethylsiloxane copolymer, vinyl-terminated diethyl siloxane-dimethylsiloxane copolymer, vinylmethylsiloxane-dimethylsiloxane copolymer, trimethylsiloxy-terminated vinylmethylsiloxane-dimethylsiloxane copolymer, silanol-terminated vinylmethylsiloxane-dimethylsiloxane copolymer, vinylmethylsiloxane homopolymer, vinyl T-structure polymer, monovinyl-terminated polydimethylsiloxane, vinylmethylsiloxane terpolymer, and vinylmethoxysilane homopolymer.

4. The composition according to claim 1, wherein the polymer B is at least one selected from the group consisting of hydride-terminated polydimethylsiloxane, hydride-terminated polyphenyl-(dimethylhydrosiloxy)siloxane, hydride-terminated methylhydrosiloxane-phenylmethylsiloxane copolymer, trimethylsiloxy-terminated methylhydrosiloxane-dimethylsiloxane copolymer, polymethylhydrosiloxane, trimethylsiloxy-terminated polyethylhydrosiloxane, triethylsiloxane, methylhydrosiloxane-phenyloctylmethylsiloxane copolymer, and methylhydrosiloxane-phenyloctylmethylsiloxane terpolymer.

5. The composition according to claim 1, wherein the viscosity of the first agent is 20,000 mPa·s or less.

6. The composition according to claim 1, wherein the first agent comprises at least one selected from the group consisting of a fiber, a pigment, a dye, and a filler.

7. A composition according to claim 1, wherein the second agent is free of a pigment, a dye and a filler.

8. A method for using the composition for forming an artificial skin according to claim 1, wherein
after the first agent is applied to the surface of a body to form a first agent layer, a second agent is applied on the first agent layer and cross-linked to form an artificial skin, or
after the second agent is applied to the surface of the body to form a second agent layer, the first agent is applied on the second agent layer and cross-linked to form an artificial skin.

9. The method for using according to claim 8, wherein the cross-linking time is 30 seconds or less.
